(19) United States
(12) Patent Application Publication
(10) Pub. No.: US 2026/0207293 A1
(43) Pub. Date: Jul. 23, 2026

(54) DRAPE PLATE ASSEMBLY

(71) Applicant: Vicarious Surgical Inc., Waltham, MA (US)

(72) Inventors: Alexander Breton, Cambridge, MA (US); Alberto Pimentel, Waltham, MA (US)

(21) Appl. No.: 19/144,132

(22) PCT Filed: Dec. 28, 2023
(86) PCT No.: PCT/US2023/086143

§ 371 Date: Jun. 27, 2025

Related U.S. Application Data
(60) Provisional application No. 63/435,696, filed on Dec. 28, 2022.

(51) Int. Cl. A61B 90/40; A61B 34/30

(52) U.S. Cl. CPC A61B 90/40; A61B 34/30

(57) ABSTRACT

A drape plate assembly for a mechanical drive of a surgical robotic device including a frame assembly, a plate assembly, and a plurality of disks disposed within the plate assembly. A motor drive system for a surgical robotic system including a drive unit and the drape plate assembly. A method of draping the surgical robotic device including attached the drape plate assembly to the surgical robotic device, providing a drape film and heat sealing one or more edges of the drape film to the drape plate assembly.

### CROSS-REFERENCE TO RELATED APPLICATIONS

This application claims the benefit of U.S. Provisional Ser. No. 63/435,696, filed Dec. 28, 2022, the entire contents of which are incorporated by reference herein in their entirety.

### BACKGROUND OF THE DISCLOSURE

Surgical robotic systems permit a surgeon (also described herein as an “operator” or a “user”) to perform an operation using robotically-controlled instruments to perform tasks and functions during a procedure.

### SUMMARY

The present disclosure is directed to a drape plate assembly for a mechanical drive of a surgical robotic device. The drape plate assembly may form part of a sterile barrier (also referred to as a drape) between non-consumable components of the surgical robotic device (which may also be referred to as capital equipment) and a patient during a procedure. For example, the drape plate assembly may provide a sterile barrier between a cassette containing spooleys that control instruments or tools of the surgical robotic device and a drive unit of the surgical robotic device.

The drape plate assembly may include a frame assembly having a first portion mateable to a second portion. The drape plate assembly may include a plate assembly having a first portion mateable to a second portion, each of the first portion and the second portion of the plate assembly having a plurality of apertures. The plate assembly is disposable between the first portion and the second portion of the frame assembly. The drape plate assembly may include a plurality of disks disposable between the first portion and the second portion of the plate assembly, each of the plurality of disks disposable in a respective one of the plurality of apertures in the first portion of the plate assembly and in a respective one of the plurality of apertures in the second portion of the plate assembly.

In some embodiments, the first portion of the plate assembly includes one or more bosses on a surface of the first portion of the plate assembly facing the first portion of the frame assembly. In some embodiments, the first portion of the frame assembly includes a first channel on a first longitudinal side of the first portion of the frame assembly and a second channel on a second longitudinal side of the first portion of the frame assembly that is opposite the first longitudinal side, and wherein the first channel and the second channel allow slidable mating with a cassette.

In some embodiments, the drape plate assembly further includes an interface circuit board mounted to the frame assembly and configured to couple with a second interface circuit board of the surgical robotic device. In some embodiments, the drape plate assembly further includes a plurality of springs disposed between the first portion of the plate assembly and the second portion of the frame assembly permitting compression of the plate assembly within the frame assembly.

In some embodiments, each of the plurality of disks includes a first hub on a first side of the disk, a second hub on a second side of the disk opposite the first side and a collar extending radially outward between the first side of the disk and the second side of the disk. The first hub includes a first mating feature, and the second hub includes a second mating feature.

In some embodiments, each of the plurality of disks is configured to engage with a motor coupling of a drive unit of the surgical robotic device. In some embodiments, each of the motor couplings comprises a coupling crown with a coupling crown mating feature that couples with the first mating feature of the first hub. In some embodiments, the collar has a diameter greater than either of a first hub diameter of the first hub and a second hub diameter of the second hub.

In some embodiments, each of the apertures in the first portion of the plate assembly has a first diameter that is smaller than the diameter of the collar and larger than the first hub diameter. Each of the plurality of apertures in the second portion of the plate assembly has a second diameter that is smaller than the diameter of the collar and larger than the second hub diameter. In some embodiments, the first diameter and the first hub diameter are selected so that the disk may rotate freely within the plate assembly while minimizing a gap between the disk and the plate assembly. In some embodiments, the plate assembly allows limited axial movement of each of the plurality of disks and allows unlimited rotational movement of each of the plurality of disks while holding the disks between the first portion and the second portion of the plate assembly. In some embodiments, the second diameter and the second hub diameter are selected so that the disk may rotate freely within the plate assembly while minimizing a gap between the disk and the plate assembly.

In some embodiments, the first mating feature includes a first rectangularly shaped bar extending along a surface of the first hub through a central portion thereof, and the second mating feature includes a second rectangularly shaped bar extending along a surface of the second hub through a central portion thereof.

In some embodiments, the first bar and the second bar are offset from each other at approximately ninety degrees. In some embodiments, the first bar includes one or more chamfered edges.

In some embodiments, a connector is mounted to the frame assembly and configured to electrically couple a first portion of the surgical robotic device with a second portion.

The present disclosure is directed to a motor drive system for a surgical robotic system including a drive unit and a drape plate assembly as described above. The drive unit may include, a drive unit housing, a plurality of drive motors within the drive unit housing, a motor mount plate, and a plurality of motor couplings.

The present disclosure is directed to a method of draping a surgical robotic device.

The method can be performed by, attaching a drape plate assembly as taught herein to a drive unit of the surgical robotic device. Providing drape film over one or more components of the surgical robotic device, and heat sealing one or more edges of the drape film to the frame assembly of the drape plate assembly.

### BRIEF DESCRIPTION OF THE DRAWINGS

These and other features and advantages of the present invention will be more fully understood by reference to the following detailed description in conjunction with the attached drawings in which like reference numerals refer to like elements throughout the different views. The drawings illustrate principals of the invention and, although not to scale, show relative dimensions.

FIG. 1 schematically depicts a surgical robotic system in accordance with some embodiments.

FIG. 2A is a perspective view of a patient cart including a robotic support system coupled to a robotic subsystem of the surgical robotic system in accordance with some embodiments.

FIG. 2B is a perspective view of an example operator console of a surgical robotic system of the present disclosure in accordance with some embodiments.

FIG. 3A schematically depicts a side view of a surgical robotic system performing a surgery within an internal cavity of a subject in accordance with some embodiments.

FIG. 3B schematically depicts a top view of the surgical robotic system performing the surgery within the internal cavity of the subject of FIG. 3A in accordance with some embodiments.

FIG. 4A is a perspective view of a single robotic arm subsystem in accordance with some embodiments.

FIG. 4B is a perspective side view of a single robotic arm of the single robotic arm subsystem of FIG. 4A in accordance with some embodiments.

FIG. 5 is a perspective front view of a camera assembly and a robotic arm assembly in accordance with some embodiments.

FIG. 6A is a perspective side view of a draped robot support subsystem in accordance with some embodiments.

FIG. 6B is a perspective side view of a draped positioning arm housing with a drive unit, drape plate, and cassette in accordance with some embodiments.

FIG. 7A is a perspective view of a drive unit in accordance with some embodiments.

FIG. 7B is a perspective cutaway view of a drive unit in accordance with some embodiments.

FIG. 8A is a perspective view of a portion of a drive motor and a motor coupling in accordance with some embodiments.

FIG. 8B is a perspective view of a portion of a drive motor and a motor coupling in accordance with some embodiments.

FIG. 9A is a top perspective view of a drape plate assembly in accordance with some embodiments.

FIG. 9B is a bottom perspective view of a drape plate assembly in accordance with some embodiments.

FIG. 9C is a side cross sectional view of a drape plate assembly in accordance with some embodiments.

FIG. 9D is an end view of a drape plate assembly in accordance with some embodiments.

FIG. 10A is a cutaway view of a portion of a drape plate assembly in accordance with some embodiments.

FIG. 10B is an exploded view of a drape plate assembly in accordance with some embodiments.

FIG. 11 is a perspective view of a coupling crown in accordance with some embodiments.

FIG. 12A is a perspective view of a disk in accordance with some embodiments.

FIG. 12B is a perspective view of a disk in accordance with some embodiments.

FIG. 13 is a perspective view of a drape plate assembly attached to a motor mount plate of the drive unit in accordance with some embodiments.

FIG. 14A is a perspective view of a cassette partially mounted to a drape plate assembly when the drape plate assembly is mounted on the drive unit.

FIG. 14B is a perspective view of a cassette mounted to a drape plate assembly when the drape plate assembly is mounted on the drive unit.

FIG. 15A is a perspective view of a cassette partially mounted to a drape plate assembly.

FIG. 15B is a perspective view of a cassette partially mounted to a drape plate assembly.

FIG. 15C is a perspective view of a cassette mounted to a drape plate assembly.

### DETAILED DESCRIPTION

Embodiments taught herein provide drape plates for surgical robotic devices and methods of draping surgical robotic devices to provide a sterile barrier between portions of the surgical robotic devices and patients during a procedure.

Advantages of some embodiments employing the drape plates and methods of draping taught herein may include more convenient and more thorough draping of the surgical robotic device. A further advantage of some embodiments is the ability to maintain drive of the robotic tools or instruments through the sterile barrier and to facilitate maintaining the barrier while attaching components to the surgical robotic device.

While various embodiments of devices, systems, and methods for a drape plate assembly for a surgical robotic system are illustrated and described herein, it will be clear to those skilled in the art that such embodiments are provided by way of example. Numerous variations, changes, and substitutions may occur to those skilled in the art without departing from the invention. It may be understood that various alternatives to the embodiments of the invention described herein may be employed. For convenience, like reference numbers are used to reference similar features of the various embodiments shown in the figures, unless otherwise noted.

As used in the specification and claims, the singular form “a,” “an,” and “the” include plural references unless the context clearly dictates otherwise. It will be further understood that the terms “comprises” and/or “comprising,” or “include” and/or “including,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.” Although some exemplary embodiments may be described herein or in documents incorporated by reference as employing a plurality of units to perform exemplary processes, it is understood that exemplary processes may also be performed by one or a plurality of modules. Additionally, it is understood that the term controller/control unit may refer to a hardware device that includes a memory and a processor and is specifically programmed to execute the processes described herein in accordance with some embodiments. In some embodiments, the memory is configured to store the modules and the processor is specifically configured to execute said modules to perform one or more processes which are described further below. In some embodiments, multiple different controllers or control units or multiple different types of controllers or control units may be employed in performing one or more processes. In some embodiments, different controllers or control units may be implemented in different portions of a surgical robotic system.

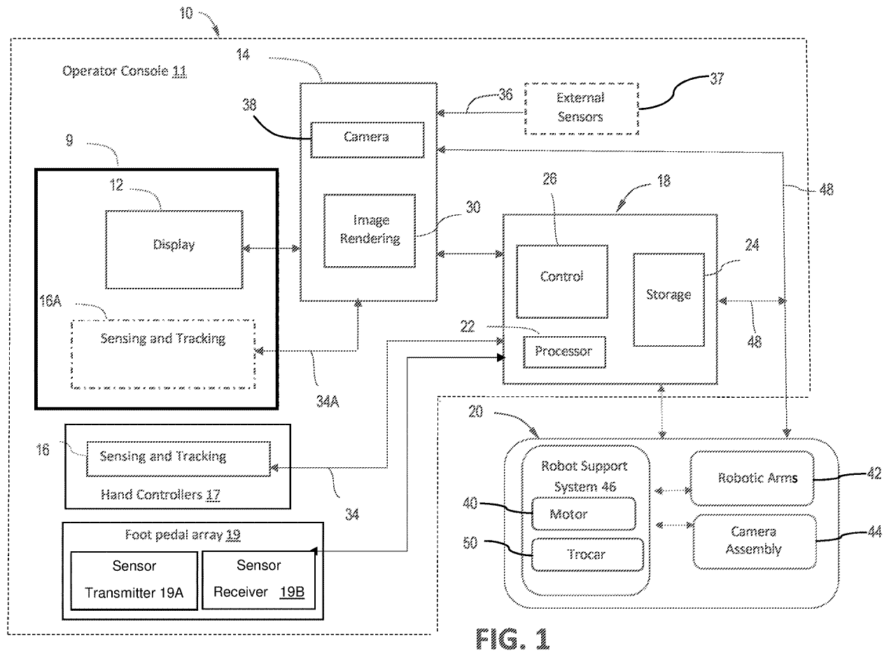

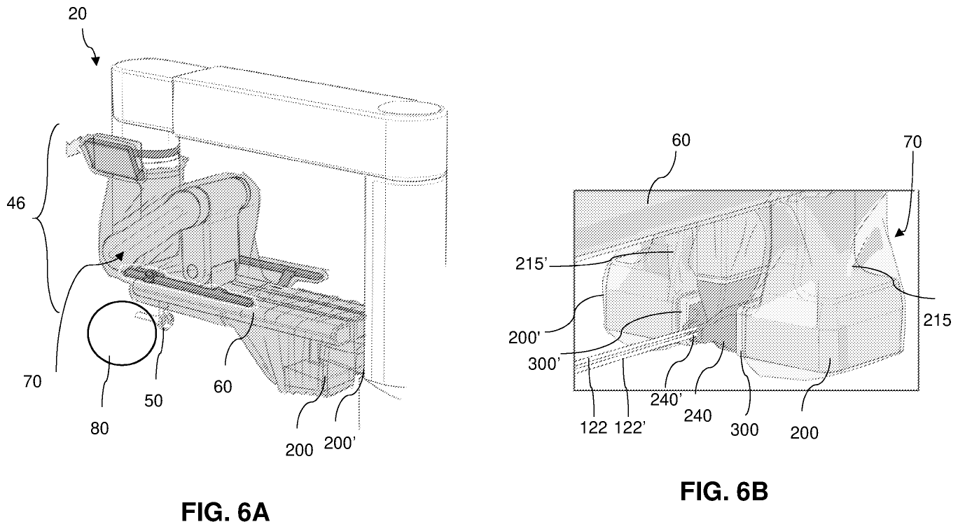

Prior to providing additional specific description of the drape plate assembly and methods of draping with respect to FIGS. 6A-18B, a surgical robotic system in which some embodiments could be employed is described below with respect to FIGS. 1-5.

### Surgical Robotic Systems

Some embodiments may be employed with a surgical robotic system. A system for robotic surgery may include a robotic subsystem. The robotic subsystem includes at least a portion, which may also be referred to herein as a robotic assembly that can be inserted into a patient via a trocar through a single incision point or site. The portion inserted into the patient via a trocar is small enough to be deployed in vivo at the surgical site and is sufficiently maneuverable when inserted to be able to move within the body to perform various surgical procedures at multiple different points or sites. The portion inserted into the body that performs functional tasks may be referred to as a surgical robotic unit, a surgical robotic module or a robotic assembly herein. The surgical robotic unit or surgical robotic module can include multiple different subunits or parts that may be inserted into the trocar separately.

The surgical robotic unit, surgical robotic module or robotic assembly can include multiple separate robotic arms that are deployable within the patient along different or separate axes. These multiple separate robotic arms may be collectively referred to as a robotic arm assembly herein. Further, a surgical camera assembly can also be deployed along a separate axis. The surgical robotic unit, surgical robotic module, or robotic assembly may also include the surgical camera assembly. Thus, the surgical robotic unit, surgical robotic module, or robotic assembly employs multiple different components, such as a pair of robotic arms and a surgical or robotic camera assembly, each of which are deployable along different axes and are separately manipulatable, maneuverable, and movable. The robotic arms and the camera assembly that are disposable along separate and manipulatable axes is referred to herein as the Split Arm (SA) architecture. The SA architecture is designed to simplify and increase efficiency of the insertion of robotic surgical instruments through a single trocar at a single insertion site, while concomitantly assisting with deployment of the surgical instruments into a surgical ready state as well as the subsequent removal of the surgical instruments through the trocar. By way of example, a surgical instrument can be inserted through the trocar to access and perform an operation in vivo in the abdominal cavity of a patient. In some embodiments, various surgical instruments may be used or employed, including but not limited to robotic surgical instruments, as well as other surgical instruments known in the art.

The systems, devices, and methods disclosed herein can be incorporated into and/or used with a robotic surgical device and associated system disclosed for example in U.S. Pat. No. 10,285,765 and in PCT patent application Serial No. PCT/US2020/39203, and/or with the camera assembly and system disclosed in United States Publication No. 2019/0076199, and/or the systems and methods of exchanging surgical tools in an implantable surgical robotic system disclosed in PCT patent application Serial No.

PCT/US2021/058820, where the content and teachings of all of the foregoing patents, patent applications and publications are incorporated herein by reference herein in their entirety.

The surgical robotic unit that forms part of the present invention can form part of a surgical robotic system that includes a surgeon workstation that includes appropriate sensors and displays, and a robot support system (RSS) for interacting with and supporting the robotic subsystem of the present invention in some embodiments. The robotic subsystem includes a motor and a surgical robotic unit that includes one or more robotic arms and one or more camera assemblies in some embodiments. The robotic arms and camera assembly can form part of a single support axis robotic system, can form part of the split arm (SA) architecture robotic system, or can have another arrangement. The robot support system can provide multiple degrees of freedom such that the robotic unit can be maneuvered within the patient into a single position or multiple different positions. In one embodiment, the robot support system can be directly mounted to a surgical table or to the floor or ceiling within an operating room. In another embodiment, the mounting is achieved by various fastening means, including but not limited to, clamps, screws, or a combination thereof. In other embodiments, the structure may be free standing. The robot support system can mount a motor assembly that is coupled to the surgical robotic unit, which includes the robotic arms and the camera assembly. The motor assembly can include gears, motors, drivetrains, electronics, and the like, for powering the components of the surgical robotic unit.

The robotic arms and the camera assembly are capable of multiple degrees of freedom of movement. According to some embodiments, when the robotic arms and the camera assembly are inserted into a patient through the trocar, they are capable of movement in at least the axial, yaw, pitch, and roll directions. The robotic arms are designed to incorporate and employ a multi-degree of freedom of movement robotic arm with an end effector mounted at a distal end thereof that corresponds to a wrist area or joint of the user. In other embodiments, the working end (e.g., the end effector end) of the robotic arm is designed to incorporate and use or employ other robotic surgical instruments, such as for example the surgical instruments set forth in U.S. Publ. No. 2018/0221102, the entire contents of which are herein incorporated by reference.

Turning to the drawings, FIG. 1 is a schematic illustration of an example surgical robotic system 10 in which aspects of the present disclosure can be employed in accordance with some embodiments of the present disclosure. The surgical robotic system 10 includes an operator console 11 and a robotic subsystem 20 in accordance with some embodiments.

The operator console 11 includes a visualization system 9 with a display device 12, an image computer 14, which may be a three-dimensional (3D) computer, hand controllers 17 having a sensor and tracker 16, and a computer 18. Additionally, the operator console 11 may include a foot pedal array 19 including a plurality of pedals. The foot pedal array 19 may include a sensor transmitter 19A and a sensor receiver 19B to sense presence of a user's foot proximate foot pedal array 19.

The display 12 may be any selected type of display for displaying information, images or video generated by the image computer 14, the computer 18, and/or the robotic subsystem 20. The visualization system 9 can include or form part of, for example, a head-mounted display (HMD), an augmented reality (AR) display (e.g., an AR display, or AR glasses in combination with a screen or display), a screen or a display, a two-dimensional (2D) screen or display, a three-dimensional (3D) screen or display, and the like. The visualization system 9 can also include an optional sensor and tracker 16A. In some embodiments, the display 12 can include an image display for outputting an image from a camera assembly 44 of the robotic subsystem 20. Discussed in more detail below is a fluid cooled camera assembly 244 suitable for use in place of the camera assembly 44.

In some embodiments, if the visualization system 9 includes an HMD device, an AR device that senses head position, or another device that employs an associated sensor and tracker 16A, the HMD device or head tracking device generates tracking and position data 34A that is received and processed by image computer 14. In some embodiments, the HMD, AR device, or other head tracking device can provide an operator (e.g., a surgeon, a nurse or other suitable medical professional) with a display that is at least in part coupled or mounted to the head of the operator, lenses to allow a focused view of the display, and the sensor and tracker 16A to provide position and orientation tracking of the operator's head. The sensor and tracker 16A can include for example accelerometers, gyroscopes, magnetometers, motion processors, infrared tracking, eye tracking, computer vision, emission and sensing of alternating magnetic fields, and any other method of tracking at least one of position and orientation, or any combination thereof. In some embodiments, the HMD or AR device can provide image data from the camera assembly 44 to the right and left eyes of the operator. In some embodiments, in order to maintain a virtual reality experience for the operator, the sensor and tracker 16A, can track the position and orientation of the operator's head, generate tracking and position data 34A, and then relay the tracking and position data 34A to the image computer 14 and/or the computer 18 either directly or via the image computer 14.

The hand controllers 17 are configured to sense a movement of the operator's hands and/or arms to manipulate the surgical robotic system 10. The hand controllers 17 can include the sensor and tracker 16, circuity, and/or other hardware. The sensor and tracker 16 can include one or more sensors or detectors that sense movements of the operator's hands.

In some embodiments, the one or more sensors or detectors that sense movements of the operator's hands are disposed in a pair of hand controllers that are grasped by or engaged by hands of the operator. In some embodiments, the one or more sensors or detectors that sense movements of the operator's hands are coupled to the hands and/or arms of the operator. For example, the sensors of the sensor and tracker 16 can be coupled to a region of the hand and/or the arm, such as the fingers, the wrist region, the elbow region, and/or the shoulder region. If the HMD is not used, then additional sensors can also be coupled to a head and/or neck region of the operator in some embodiments. If the operator employs the HMD, then the eyes, head and/or neck sensors and associated tracking technology can be built-in or employed within the HMD device, and hence form part of the optional sensor and tracker 16A as described above. In some embodiments, the sensor and tracker 16 can be external and coupled to the hand controllers 17 via electricity components and/or mounting hardware. In some embodiments, the optional sensor and tracker 16A may sense and track movement of one or more of an operator's head, of at least a portion of an operator's head, an operator's eyes or an operator's neck based, at least in part, on imaging of the operator in addition to or instead of by a sensor or sensors attached to the operator's body.

In some embodiments, the sensor and tracker 16 can employ sensors coupled to the torso of the operator or any other body part. In some embodiments, the sensor and tracker 16 can employ in addition to the sensors an Inertial Momentum Unit (IMU) having for example an accelerometer, gyroscope, magnetometer, and a motion processor. The addition of a magnetometer allows for reduction in sensor drift about a vertical axis. In some embodiments, the sensor and tracker 16 also include sensors placed in surgical material such as gloves, surgical scrubs, or a surgical gown. The sensors can be reusable or disposable. In some embodiments, sensors can be disposed external of the operator, such as at fixed locations in a room, such as an operating room. The external sensors 37 can generate external data 36 that can be processed by the computer 18 and hence employed by the surgical robotic system 10.

The sensors generate position and/or orientation data indicative of the position and/or orientation of the operator's hands and/or arms. The sensor and tracker 16 16 and/or 16A can be utilized to control movement (e.g., changing a position and/or an orientation) of the camera assembly 44 and robotic arms 42 of the robotic subsystem 20. The tracking and position data 34 generated by the sensor and tracker 16 can be conveyed to the computer 18 for processing by at least one processor 22.

The computer 18 can determine or calculate, from the tracking and position data 34 and 34A, the position and/or orientation of the operator's hands or arms, and in some embodiments of the operator's head as well, and convey the tracking and position data 34 and 34A to the robotic subsystem 20. The tracking and position data 34, 34A can be processed by the processor 22 and can be stored for example in the storage 24. The tracking and position data 34 and 34A can also be used by the controller 26, which in response can generate control signals for controlling movement of the robotic arms 42 and/or the camera assembly 44. For example, the controller 26 can change a position and/or an orientation of at least a portion of the camera assembly 44, of at least a portion of the robotic arms 42, or both. In some embodiments, the controller 26 can also adjust the pan and tilt of the camera assembly 44 to follow the movement of the operator's head.

The robotic subsystem 20 can include a robot support system (RSS) 46 having a motor 40 and a trocar 50 or trocar mount, the robotic arms 42, and the camera assembly 44. The robotic arms 42 and the camera assembly 44 can form part of a single support axis robot system, such as that disclosed and described in U.S. Pat. No. 10,285,765, or can form part of a split arm (SA) architecture robot system, such as that disclosed and described in PCT Patent Application No. PCT/US2020/039203, both of which are incorporated herein by reference in their entirety.

The robotic subsystem 20 can employ multiple different robotic arms that are deployable along different or separate axes. In some embodiments, the camera assembly 44, which can employ multiple different camera elements, can also be deployed along a common separate axis. Thus, the surgical robotic system 10 can employ multiple different components, such as a pair of separate robotic arms and the camera assembly 44, which are deployable along different axes. In some embodiments, the robotic arms 42 and the camera assembly 44 are separately manipulatable, maneuverable, and movable. The robotic subsystem 20, which includes the robotic arms 42 and the camera assembly 44, is disposable along separate manipulatable axes, and is referred to herein as an SA architecture. The SA architecture is designed to simplify and increase efficiency of the insertion of robotic surgical instruments through a single trocar at a single insertion point or site, while concomitantly assisting with deployment of the surgical instruments into a surgical ready state, as well as the subsequent removal of the surgical instruments through a trocar 50 as further described below.

The RSS 46 can include the motor 40 and the trocar 50 or a trocar mount. The RSS 46 can further include a support member that supports the motor 40 coupled to a distal end thereof. The motor 40 in turn can be coupled to the camera assembly 44 and to each of the robotic arms 42. The support member can be configured and controlled to move linearly, or in any other selected direction or orientation, one or more components of the robotic subsystem 20. In some embodiments, the RSS 46 can be free standing. In some embodiments, the RSS 46 can include the motor 40 that is coupled to the robotic subsystem 20 at one end and to an adjustable support member or element at an opposed end.

The motor 40 can receive the control signals generated by the controller 26. The motor 40 can include gears, one or more motors, drivetrains, electronics, and the like, for powering and driving the robotic arms 42 and the cameras assembly 44 separately or together. The motor 40 can also provide mechanical power, electrical power, mechanical communication, and electrical communication to the robotic arms 42, the camera assembly 44, and/or other components of the RSS 46 and robotic subsystem 20. The motor 40 can be controlled by the computer 18. The motor 40 can thus generate signals for controlling one or more motors that in turn can control and drive the robotic arms 42, including for example the position and orientation of each articulating joint of each robotic arm, as well as the camera assembly 44. The motor 40 can further provide for a translational or linear degree of freedom that is first utilized to insert and remove each component of the robotic subsystem 20 through a trocar 50. The motor 40 can also be employed to adjust the inserted depth of each robotic arm 42 when inserted into the patient 100 through the trocar 50.

The trocar 50 is a medical device that can be made up of an awl (which may be a metal or plastic sharpened or non-bladed tip), a cannula (essentially a hollow tube), and a seal in some embodiments. The trocar can be used to place at least a portion of the robotic subsystem 20 in an interior cavity of a subject (e.g., a patient) and can withdraw gas and/or fluid from a body cavity. The robotic subsystem 20 can be inserted through the trocar to access and perform an operation in vivo in a body cavity of a patient. In some embodiments, the robotic subsystem 20 can be supported, at least in part, by the trocar 50 or a trocar mount with multiple degrees of freedom such that the robotic arms 42 and the camera assembly 44 can be maneuvered within the patient into a single position or multiple different positions. In some embodiments, the robotic arms 42 and camera assembly 44 can be moved with respect to the trocar 50 or a trocar mount with multiple different degrees of freedom such that the robotic arms 42 and the camera assembly 44 can be maneuvered within the patient into a single position or multiple different positions.

In some embodiments, the RSS 46 can further include an optional controller for processing input data from one or more of the system components (e.g., the display 12, the sensor and tracker 16, the robotic arms 42, the camera assembly 44, and the like), and for generating control signals in response thereto. The motor 40 can also include a storage element for storing data in some embodiments.

The robotic arms 42 can be controlled to follow the scaled-down movement or motion of the operator's arms and/or hands as sensed by the associated sensors in some embodiments and in some modes of operation. The robotic arms 42 include a first robotic arm including a first end effector at distal end of the first robotic arm, and a second robotic arm including a second end effector disposed at a distal end of the second robotic arm. In some embodiments, the robotic arms 42 can have portions or regions that can be associated with movements associated with the shoulder, elbow, and wrist joints as well as the fingers of the operator. For example, the robotic elbow joint can follow the position and orientation of the human elbow, and the robotic wrist joint can follow the position and orientation of the human wrist. The robotic arms 42 can also have associated therewith end regions that can terminate in end-effectors that follow the movement of one or more fingers of the operator in some embodiments, such as for example the index finger as the user pinches together the index finger and thumb. In some embodiments, while the robotic arms 42 may follow movement of the arms of the operator in some modes of control while a virtual chest of the robotic assembly may remain stationary (e.g., in an instrument control mode). In some embodiments, the position and orientation of the torso of the operator are subtracted from the position and orientation of the operator's arms and/or hands. This subtraction allows the operator to move his or her torso without the robotic arms moving. Further disclosure regarding control of movement of individual arms of a robotic arm assembly is provided in International Patent Application Publications WO 2022/094000 A1 and WO 2021/231402 A1, each of which is incorporated by reference herein in its entirety.

The camera assembly 44 is configured to provide the operator with image data 48, such as for example a live video feed of an operation or surgical site, as well as enable the operator to actuate and control the cameras forming part of the camera assembly 44. In some embodiments, the camera assembly 44 can include one or more cameras (e.g., a pair of cameras), the optical axes of which are axially spaced apart by a selected distance, known as the inter-camera distance, to provide a stereoscopic view or image of the surgical site. In some embodiments, the operator can control the movement of the cameras via movement of the hands via sensors coupled to the hands of the operator or via hand controllers grasped or held by hands of the operator, thus enabling the operator to obtain a desired view of an operation site in an intuitive and natural manner. In some embodiments, the operator can additionally control the movement of the camera via movement of the operator's head. The camera assembly 44 is movable in multiple directions, including for example in yaw, pitch and roll directions relative to a direction of view. In some embodiments, the components of the stereoscopic cameras can be configured to provide a user experience that feels natural and comfortable. In some embodiments, the interaxial distance between the cameras can be modified to adjust the depth of the operation site perceived by the operator.

The image or video data 48 generated by the camera assembly 44 can be displayed on the display 12. In embodiments in which the display 12 includes a HMD, the display can include the built-in sensor and tracker 16A that obtains raw orientation data for the yaw, pitch and roll directions of the HMD as well as positional data in Cartesian space (x, y, z) of the HMD. In some embodiments, positional and orientation data regarding an operator's head may be provided via a separate head-tracker. In some embodiments, the sensor and tracker 16A may be used to provide supplementary position and orientation tracking data of the display in lieu of or in addition to the built-in tracking system of the HMD. In some embodiments, no head tracking of the operator is used or employed. In some embodiments, images of the operator may be used by the sensor and tracker 16A for tracking at least a portion of the operator's head.

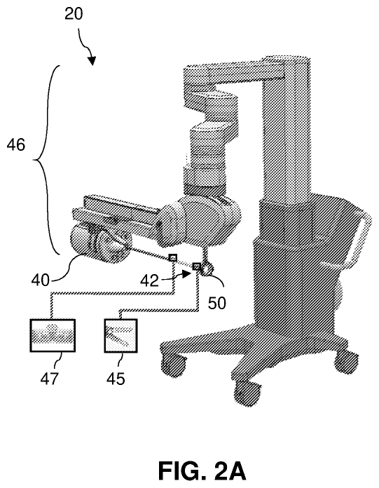

FIG. 2A depicts an example robotic assembly 20, which is also referred to herein as a robotic subsystem, of a surgical robotic system 10 incorporated into or mounted onto a mobile patient cart in accordance with some embodiments. In some embodiments, the robotic assembly 20 includes the RSS 46, which, in turn includes the motor 40, the robotic arm assembly 42 having end-effectors 45, the camera assembly 44 having one or more cameras 47, and may also include the trocar 50 or a trocar mount.

FIG. 2B depicts an example of an operator console 11 of the surgical robotic system 10 of the present disclosure in accordance with some embodiments. The operator console 11 includes the display 12, the hand controllers 17, and also includes one or more additional controllers, such as the foot pedal array 19 for control of the robotic arms 42, for control of the camera assembly 44, and for control of other aspects of the system.

FIG. 2B also depicts the left hand controller subsystem 23A and the right hand controller subsystem 23B of the operator console. The left hand controller subsystem 23A includes and supports the left hand controller 17A and the right hand controller subsystem 23B includes and supports the right hand controller 17B. In some embodiments, the left hand controller subsystem 23A may releasably connect to or engage the left hand controller 17A, and right hand controller subsystem 23B may releasably connect to or engage the right hand controller 17A. In some embodiments, the connections may be both physical and electronic so that the left hand controller subsystem 23A and the right hand controller subsystem 23B may receive signals from the left hand controller 17A and the right hand controller 17B, respectively, including signals that convey inputs received from a user selection on a button or touch input device of the left hand controller 17A or the right hand controller 17B.

Each of the left hand controller subsystem 23A and the right hand controller subsystem 23B may include components that enable a range of motion of the respective left hand controller 17A and right hand controller 17B, so that the left hand controller 17A and right hand controller 17B may be translated or displaced in three dimensions and may additionally move in the roll, pitch, and yaw directions. Additionally, each of the left hand controller subsystem 23A and the right hand controller subsystem 23B may register movement of the respective left hand controller 17A and right hand controller 17B in each of the forgoing directions and may send a signal providing such movement information to a processor (not shown) of the surgical robotic system.

In some embodiments, each of the left hand controller subsystem 23A and the right hand controller subsystem 23B may be configured to receive and connect to or engage different hand controllers (not shown). For example, hand controllers with different configurations of buttons and touch input devices may be provided. Additionally, hand controllers with a different shape may be provided. The hand controllers may be selected for compatibility with a particular surgical robotic system or a particular surgical robotic procedure or selected based upon preference of an operator with respect to the buttons and input devices or with respect to the shape of the hand controller in order to provide greater comfort and ease for the operator.

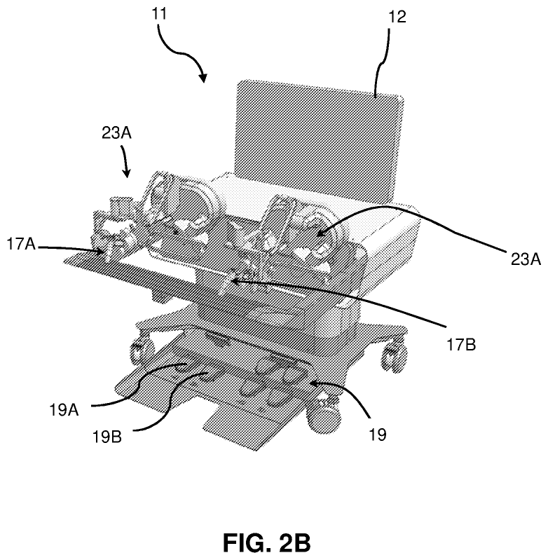

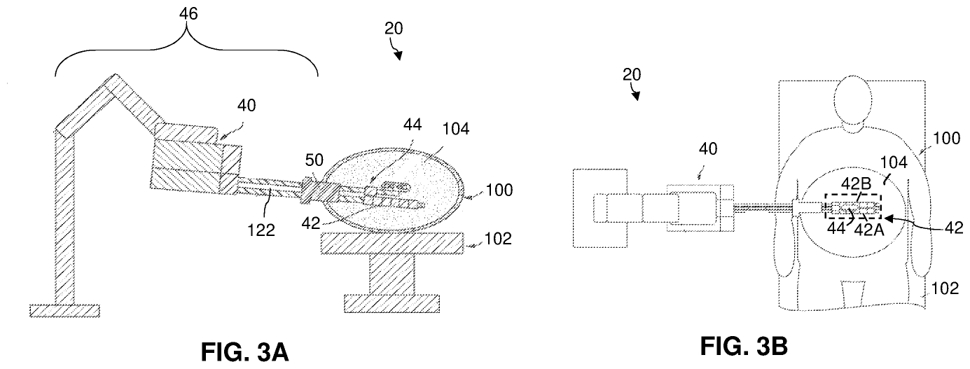

FIG. 3A schematically depicts a side view of the surgical robotic system 10 performing a surgery within an internal cavity 104 of a subject 100 in accordance with some embodiments and for some surgical procedures. FIG. 3B schematically depicts a top view of the surgical robotic system 10 performing the surgery within the internal cavity 104 of the subject 100. Robotic arm assembly 42 includes robotic arm 42A and robotic arm 42B. The subject 100 (e.g., a patient) is placed on an operation table 102 (e.g., a surgical table) . In some embodiments, and for some surgical procedures, an incision is made in the patient 100 to gain access to the internal cavity 104. The trocar 50 is then inserted into the patient 100 at a selected location to provide access to the internal cavity 104 or operation site. The RSS 46 can then be maneuvered into position over the patient 100 and the trocar 50. In some embodiments, the RSS 46 includes a trocar mount that attaches to the trocar 50. The robotic assembly 20 can be coupled to the motor 40 and at least a portion of the robotic assembly can be inserted into the trocar 50 and hence into the internal cavity 104 of the patient 100. For example, the camera assembly 44 and the robotic arm assembly 42 can be inserted individually and sequentially into the patient 100 through the trocar 50. Although the camera assembly and the robotic arm assembly may include some portions that remain external to the subject's body in use, references to insertion of the robotic arm assembly 42 and/or the camera assembly into an internal cavity of a subject and disposing the robotic arm assembly 42 and/or the camera assembly 44 in the internal cavity of the subject are referring to the portions of the robotic arm assembly 42 and the camera assembly 44 that are intended to be in the internal cavity of the subject during use. The sequential insertion method has the advantage of supporting smaller trocars and thus smaller incisions can be made in the patient 100, thus reducing the trauma experienced by the patient 100. In some embodiments, the camera assembly 44 and the robotic arm assembly 42 can be inserted in any order or in a specific order. In some embodiments, the camera assembly 44 can be followed by a first robot arm of the robotic arm assembly 42 and then followed by a second robot arm of the robotic arm assembly 42 all of which can be inserted into the trocar 50 and hence into the internal cavity 104. Once inserted into the patient 100, the RSS 46 can move the robotic arm assembly 42 and the camera assembly 44 to an operation site manually or automatically controlled by the operator console 11.

Further disclosure regarding control of movement of individual arms of a robotic arm assembly is provided in International Patent Application Publications WO 2022/094000 A1 and WO 2021/231402 A1, each of which is incorporated by reference herein in its entirety.

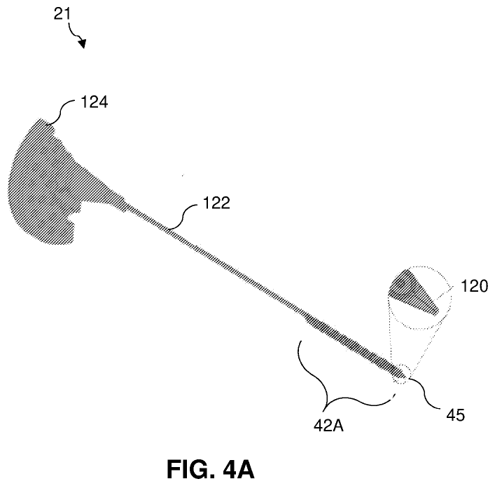

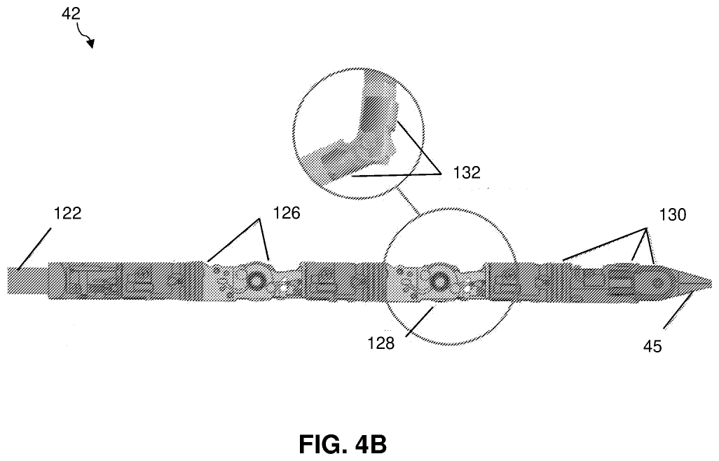

FIG. 4A is a perspective view of a robotic arm subassembly 21 in accordance with some embodiments. The robotic arm subassembly 21 includes a robotic arm 42A, the end-effector 45 having an instrument tip 120 (e.g., monopolar scissors, needle driver/holder, bipolar grasper, or any other appropriate tool), a shaft 122 supporting the robotic arm 42A. A distal end of the shaft 122 is coupled to the robotic arm 42A, and a proximal end of the shaft 122 is coupled to a housing 124 of the motor 40 (as shown in FIG. 2A). At least a portion of the shaft 122 can be external to the internal cavity 104 (as shown in FIGS. 3A and 3B). At least a portion of the shaft 122 can be inserted into the internal cavity 104 (as shown in FIGS. 3A and 3B).

FIG. 4B is a side view of the robotic arm assembly 42. The robotic arm assembly 42 includes a virtual shoulder 126, a virtual elbow 128 having position sensors 132 (e.g., capacitive proximity sensors), a virtual wrist 130, and the end-effector 45 in accordance with some embodiments. The virtual shoulder 126, the virtual elbow 128, the virtual wrist 130 can include a series of hinge and rotary joints to provide each arm with positionable, seven degrees of freedom, along with one additional grasping degree of freedom for the end-effector 45 in some embodiments.

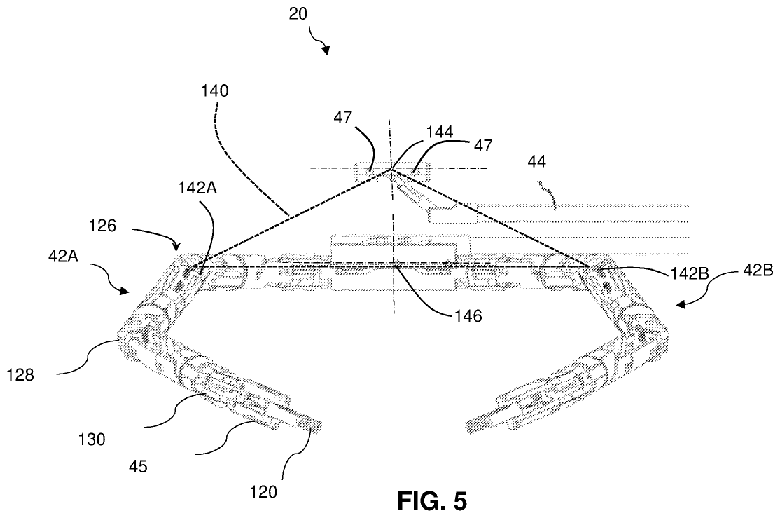

FIG. 5 illustrates a perspective front view of a portion of the robotic assembly 20 configured for insertion into an internal body cavity of a patient. The robotic assembly 20 includes a first robotic arm 42A and a second robotic arm 42B. The two robotic arms 42A and 42B can define, or at least partially define, a virtual chest 140 of the robotic assembly 20 in some embodiments. In some embodiments, the virtual chest 140 (depicted as a triangle with dotted lines) can be defined by a chest plane extending between a first pivot point 142A of a most proximal joint of the first robotic arm 42A (e.g., a shoulder joint 126), a second pivot point 142B of a most proximal joint of the second robotic arm 42B, and a camera imaging center point 144 of the camera(s) 47. A pivot center 146 of the virtual chest 140 lies in the middle of the virtual chest.

In some embodiments, sensors in one or both of the first robotic arm 42A and the second robotic arm 42B can be used by the system to determine a change in location in three-dimensional space of at least a portion of the robotic arm. In some embodiments, sensors in one or both of the first robotic arm and second robotic arm can be used by the system to determine a location in three-dimensional space of at least a portion of one robotic arm relative to a location in three-dimensional space of at least a portion of the other robotic arm.

In some embodiments, the camera assembly 44 is configured to obtain images from which the system can determine relative locations in three-dimensional space. For example, the camera assembly may include multiple cameras, at least two of which are laterally displaced from each other relative to an imaging axis, and the system may be configured to determine a distance to features within the internal body cavity. Further disclosure regarding a surgical robotic system including camera assembly and associated system for determining a distance to features may be found in International Patent Application Publication No. WO 2021/159409, entitled “System and Method for Determining Depth Perception In Vivo in a Surgical Robotic System,” and published Aug. 12, 2021, which is incorporated by reference herein in its entirety. Information about the distance to features and information regarding optical properties of the cameras may be used by a system to determine relative locations in three-dimensional space.

### Sterile Barrier or Drape

Surgical robotic devices contain a variety of components. For example, the patient cart, described in connection with FIG. 2A includes the motor 40, the robotic arm assembly 42 having end-effectors 45, the camera assembly 44 having one or more cameras 47, and may also include the trocar 50 or a trocar mount. In order to ensure sterile conditions for a patient during a procedure, a sterile barrier or drape is preferably provided between the patient and the non-consumable portions of the patient cart, which may be referred to as capital equipment. The non-consumable portions are intended to be re-used. The sterile barrier or drape helps to ensure sterile conditions for the patient and reduces any contamination of the non-consumable portions of the patient cart.

FIG. 6A illustrates the robotic subsystem 20 that may form a portion of a patient cart. The robotic subsystem 20 includes the robotic support subsystem 46, an insertion rail 60 (which may also be referred to as a positioning arm), the trocar 50, and drive units 200, 200′ (which may also be referred to as an instrument drive or a motor unit). In some embodiments, the trocar 50 forms a consumable portion that is introduced at least in part into the body of a patient (represented by a circle 80) in order to facilitate introduction of the robotic arm assembly 42 into the patient. In order to provide a sterile barrier, a drape film 70 is positioned around components of the robotic subsystem 20 including the robotic support subsystem 46, the insertion rail 60 and the drive units 200, 200′.

FIG. 6B illustrates a detailed view of the robotic subsystem 20 including the drive units 200, 200′ with shafts 122, 122′ of the robotic arm subassembly 21. The drive units 200, 200′ are connected to the position arm housing 60 by carriage attachment mounts 215, 215′. The drape film 70 covers portions of the drive units 200, 200′. The drive units 200, 200′ are connected to drape plates 300, 300′, which are in turn are connected to cassettes 240, 240′ which include, or are connected to shafts 122, 122′. Instruments connected to shafts 122, 122′ may be driven by drive units 200, 200′. In the robotic subsystem 20, a plurality of cables runs through the shafts 122, 122′ from the drive units 200, 200′ to drive instruments at the ends of shafts 122, 122′ opposite the drive units 200, 200′. The drive units 200, 200′control the plurality of cables to operate the instruments. Operation of the drive units 200, 200′ is provided below. The drape plate assemblies 300, 300′ provide a sterile barrier between the cassettes 240, 240′ and the drive units 200, 200′ and, with the drape film 70, provide a sterile barrier between non-consumable portions of the surgical robotic device 10 and the patient. As is described below in greater detail, the drape plate assemblies 300, 300′ transmit torque from the drive units 200, 200′ to the cassettes 240, 240′ while maintaining the sterile barrier.

### Drive Unit

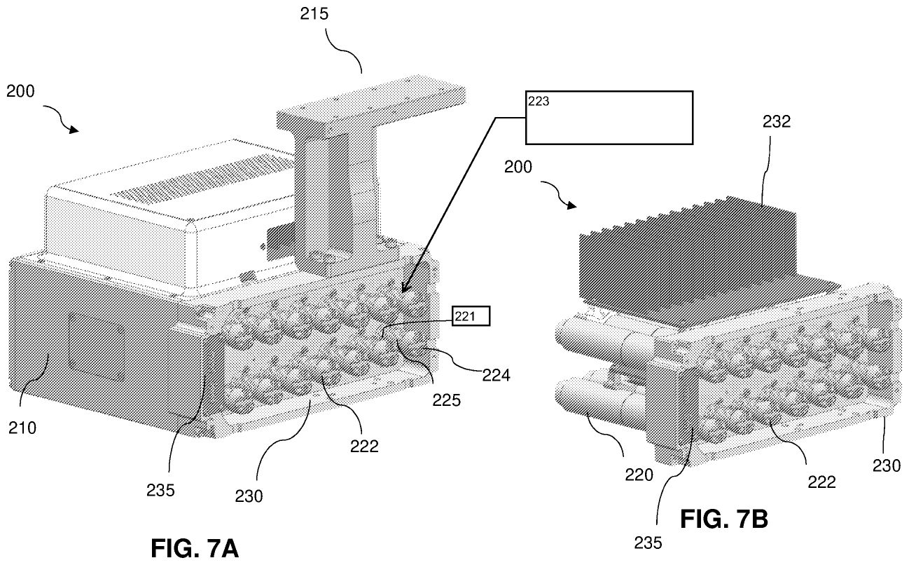

FIG. 7A illustrates the drive unit 200 including a drive unit housing 210, a carriage attachment mount 215, a motor mount plate 230, a plurality of motor couplings 222, and an interface board 235. FIG. 7B illustrates the drive unit 200 in a cutaway view showing motors 220 and servo control boards 232. The drive unit 200 may be attached to the insertion rail 60 of the robotic subsystem 20 via the carriage attachment mount 215. In some embodiments, the drive unit 200 includes fourteen motor couplings 222. Those skilled in the art will appreciate that the drive unit 200 can include less than fourteen motor couplings 222 or more than fourteen motor couplings 222. Each of the motor couplings 222 is driven by a motor 220 and may be coupled to a motor shaft 221 of the motor 220 by a coupling shaft 223. Each of the motor couplings 222 includes the coupling shaft 223, a coupling crown 224, and a spring 225. The coupling shaft 223 is connected to one of the plurality of drive motors 220, for example by connecting to the motor shaft 221, and is also connected to the coupling crown 224. The spring 225 is positioned around and coaxial with the coupling shaft 223 and biases the coupling crown 224 in an extended position. Each of the motor couplings 222 may be compressed by application of a force to the coupling crown 224. In some embodiments, more or fewer motor couplings 222 may be provided in an instrument drive 200. In some embodiments, two or more motor couplings 222 may be driven by a single motor 220. Each of the motors 220 is controlled by a servo control board 232. In some embodiments, the servo control board 232 may be consolidated or replaced with one or more motherboards.

The servo control boards 232 may be controlled via a wired connection to the interface board 235. The interface board 235 may be a disposable interface board or DIB.

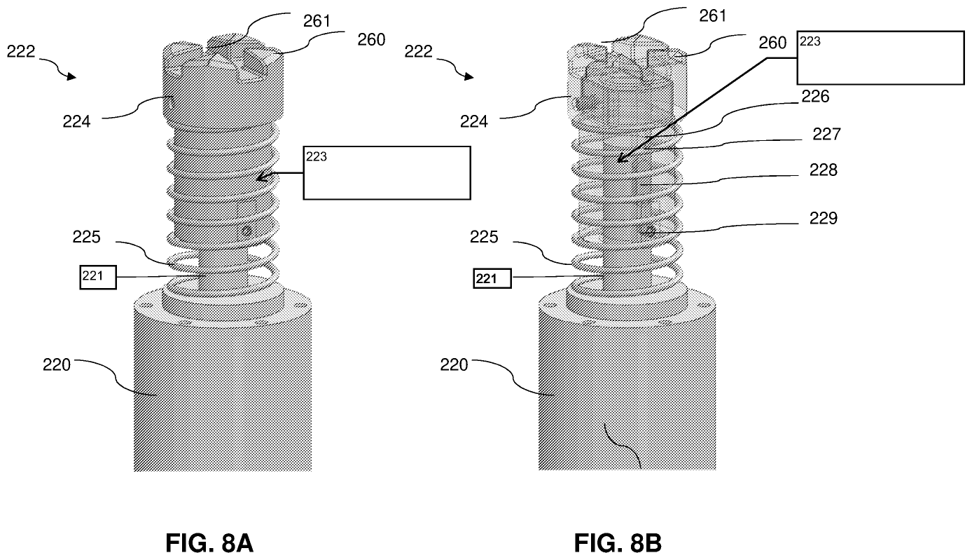

FIGS. 8A and 8B illustrate a portion of the drive motor 220 with the motor coupling 222 including the coupling shaft 223, the coupling crown 224, and the spring 225. The spring 225 is fitted coaxially with the coupling shaft 223 and the coupling crown 224 to bias the coupling crown 224 into an extended position. The coupling crown 224 has a coupling crown mating feature 260 with channels 261 to mate with a first mating feature 334 or a second mating feature 335 of a disk 330 of a drape plate assembly 300. In some embodiments, the channels 261 intersect each other at a 90 degree angle. In some embodiments, the channels 261 intersect each other at a 45 degree angle. Nonetheless, those skilled in the art will appreciate that other suitable channel intersection angles are possible.

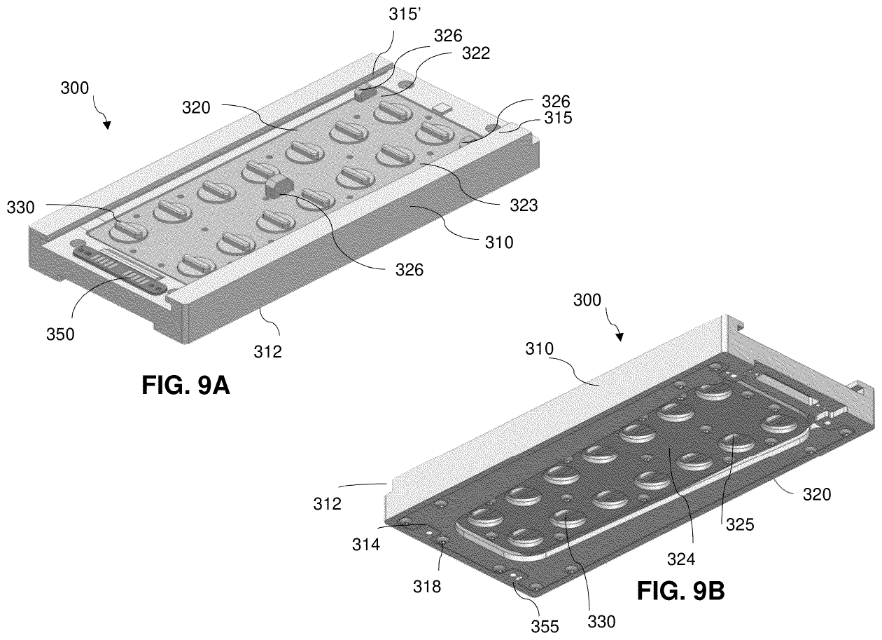

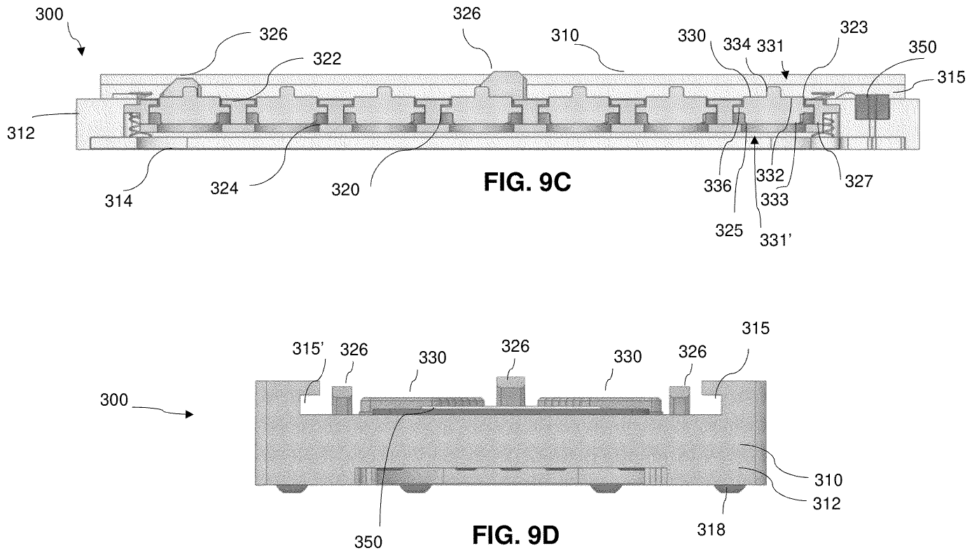

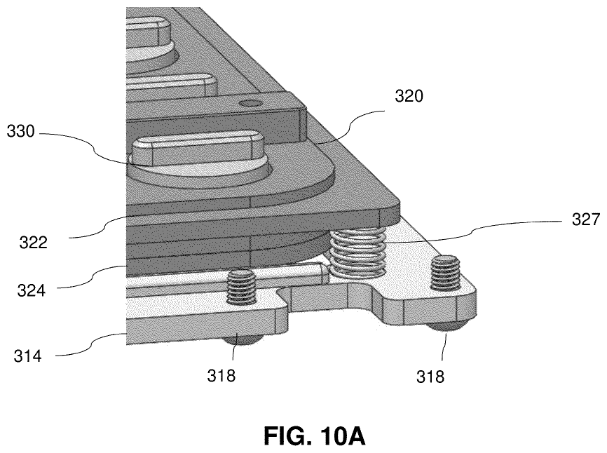

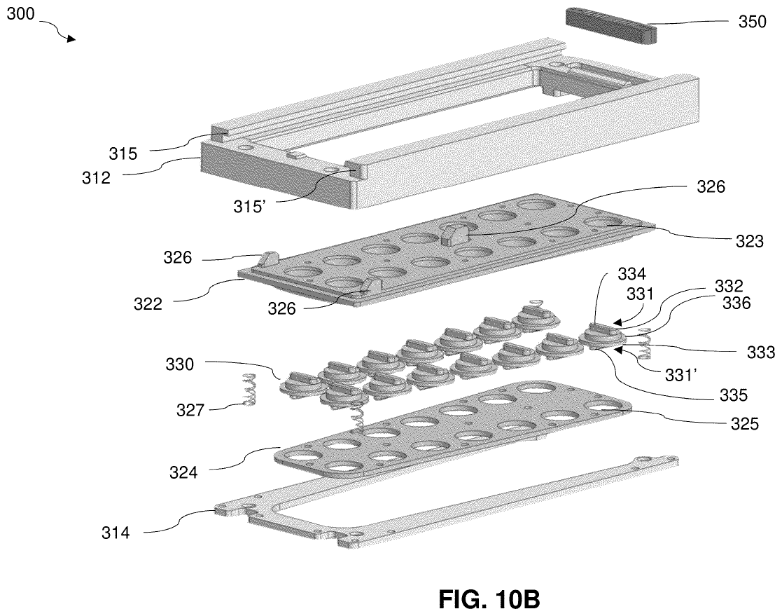

The coupling crown mating feature 260 includes one or more angled or chamfered surfaces to facilitate engagement of the first mating feature 334 or the second mating feature 335 of the disk 330 with the channels 261. The one or more angled or chamfered surfaces of the coupling crown mating feature 260 help guide one or both of the mating features 334, 335 of the disk 330 into alignment with one of the channels 261. The disk 330 is described in greater detail below. The coupling crown 224 has a pocket configured to receive the motor shaft 221. The coupling shaft 223 has an interior channel 226 (as seen in FIG. 8B) along a central longitudinal axis. The interior channel 226 forms a key way 227 that receives a corresponding key 228 of the motor coupling shaft 221. The fit of the key 228 into the key way 227 rotationally locks the coupling crown 224 and the motor coupling shaft 221 such that both turn together. However, the coupling crown 224 is able to move longitudinally with respect to the motor shaft 221 as the key 228 moves within the key way 227. This movement permits the coupling crown 224 to move from an extended position to a retracted position when force is applied to the coupling crown 224. The coupling crown 224 also includes a stop 229 extending into the key way 227. The stop 229 may be removable to permit replacement of the coupling crown 224. For example, the stop 229 may be a machine screw. The stop 229 provides a limit to the range of motion of the coupling crown 224 in the extended direction such that the coupling crown 224 does not separate from the motor shaft 221 when the stop 229 is in place. As shown in FIG. 8B, the coupling crown 224 may be of a two-piece construction. In some embodiments, the coupling crown 224 may be of a single piece construction. Accordingly, the coupling crown 224 is spring-biased in an extended position but is able to retract with a force is applied to the coupling crown 224. The drape plate assembly 300 will now be described with reference to FIGS. 9A-9D and 10. FIG. 9A illustrates a top perspective view of the drape plate assembly 300. FIG. 9B illustrates a bottom perspective view of the drape plate assembly 300. The drape plate assembly 300 includes a frame assembly 310, a plate assembly 320, and a plurality of disks 330. The frame assembly 310 includes a first portion 312 mateable to a second portion 314. The first portion 312 and the second portion 314 are connected via fasteners 318. A person of ordinary skill in the art would appreciate that the first portion 312 and the second portion 314 may be connected by various means including, for example, fasteners (e.g., rivets, machine screws), adhesives, ultrasonic welding, and snap-fit features, or frame assembly 310 may be of a unitary construction. The plate assembly 320 includes a first portion 322 mateable to a second portion 324. The first portion 322 and the second portion 324 of the plate assembly 320 include a plurality of apertures 323, 325. The plate assembly 320 is disposable between the first portion 312 and the second portion 314 of the frame assembly 310. The plurality of disks 330 are disposable between the first portion 322 and the second portion 324 of the plate assembly 320. Each of the plurality of disks 330 are disposable in a respective one of the plurality of apertures 323 in the first portion 322 of the plate assembly 320 and in a respective one of the plurality of apertures 325 in the second portion 324 of the plate assembly 320. The drape plate assembly 300 includes a plurality of springs 327 disposed between the plate assembly 320 and the second portion 314 of the frame assembly 310 permitting compression of the plate assembly 320 within the frame assembly 310. The springs 327 are held between the second portion 314 of the frame assembly 310 and the first portion 322 of the plate assembly 320, as described below in connection with FIG. 10A.

The first portion 312 of the frame assembly 310 includes a first channel 315 on a first longitudinal side of the first portion 312 of the frame assembly 310 and a second channel 315′ on a second longitudinal side of the first portion 312 of the frame assembly 310 that is opposite the first longitudinal side of the first portion 312. The first channel 315 and the second channel 315′ allow slidable mating of the cassette 240 with the drape plate assembly 300 The first portion 322 of the plate assembly 320 includes one or more bosses 326 on a surface of the first portion 322 of the plate assembly 320 facing the first portion 312 of the frame assembly 310. As the cassette 240 is attached to the drape plate assembly 300, for example, by being slid onto the drape plate assembly 300 via the first and second channel 315, 315′, the cassette 240 may engage the boss 326 to depress the plate assembly 320 which may, in turn, depress the plurality of motor couplings 222 to provide access for the cassette 240.

The drape plate assembly 300 also includes an electrical connector 350 mounted to the frame assembly 310. The electrical connector 350 includes conductive paths to operatively couple one portion of an electronic circuit to another portion of the electronic circuit or to electrically connect one electronic circuit to another electronic circuit. For example, a connector 350 mounted to the frame assembly 310 may electrically couple a first portion of the surgical robotic device 10 to a second portion. In some embodiments, the electrical connector 350 or a separate interface circuit board is configured to couple with the interface board 235 of the drive unit 200. In some embodiments, the electrical connector 350 or a separate interface circuit board is configured to couple with an interface circuit board of the cassette 240. In some embodiments, the electrical connector may allow data from instruments to pass through to the drive unit 200. In some embodiments, the drape plate assembly 300 includes an interface circuit board in place of or in conjunction with the electrical connector 350. The interface circuit board may be configured to couple with a second interface circuit board of the surgical device 10.

The drape plate assembly 300 includes a drape film connection area 355 of the frame assembly 310. In some embodiments, the drape film 70 is attached to the drape plate assembly 300 at the drape film connection area 355 via, for example, heat sealing or an adhesive. The drape film connection area 355 is on a surface of the second portion 314 of the frame assembly 310 that faces the drive unit 200 when the drape plate assembly 300 is attached to the drive unit 200. In some embodiments, the surgical robotic device 10 is draped by attaching the drape film 70 to the drape film connection area 355 of the drape plate assembly 300, then draping the drape film 70 over components of the surgical robotic device 10 and attaching the drape plate assembly 300 to the drive unit 200 of the surgical robotic device 10. In some embodiments, the surgical robotic device 10 is draped by draping the drape film 70 over one or more components of the surgical robotic device 10, then attaching the drape film 70 to the drape film connection area 355 of the drape plate assembly 300, then attaching the drape plate assembly 300 to the drive unit 200 of the surgical robotic device 10. In some embodiments, this drape method provides a continuous sterile barrier formed from the drape film 70 and the drape plate assembly 300. As shown in, for example, FIG. 6B, this draped arrangement provides a sterile barrier between the patient and components of the surgical robotic device 10 such as, for example, the drive unit 200 and the insertion rail 60.

A cross sectional view of the disk 330 is illustrated in FIG. 9C that includes a collar 336 of the disk 330.

Each of the disks 330 includes a first side 331, a second side 331′ opposite to the first side 331, and a collar 336 between the first side 331 and the second side 331′. The first side 331 includes a first hub 332 and a first mating feature 334 and the second side 331′ includes a second hub 333 and a second mating feature 335. The collar 336 has a diameter greater than either of a first hub diameter of the first hub 332 and a second hub diameter of the second hub 333. The apertures 323 in the first portion 322 of the plate assembly 320 each have a first diameter that is smaller than the diameter of the collar 336 and larger than the first hub 332 diameter and each of the plurality of apertures 325 in the second portion 324 of the plate assembly 320 has a second diameter that is smaller than the diameter of the collar 336 and larger than the second hub 333 diameter. As a result, the disks 330 may be held in the apertures 323, 325 of the first portion 322 and the second portion 324 of the plate assembly 320, with the first hub 332 placed in an aperture 323 of the first portion 322 and the second hub 333 placed in a corresponding aperture, for example, aperture 325 of the second portion 324 and the collar 336 is held between the first portion 322 and the second portion 324 of the plate assembly 320. This arrangement permits the disks 330 to rotate within the plate assembly 320 and permits the disks 330 and the plate assembly 320 to move laterally while continuing to retain the disks 330 within the plate assembly 320. For example, the first diameter of the apertures 323 of the first portion 322 of the plate assembly 320 and the first hub 332 diameter are selected so that the disk 330 may rotate freely within the plate assembly 320 while minimizing a gap between the disk 330 and the plate assembly 320 to provide a sterile barrier and further hold disk 330 in place. Similarly, the second diameter of the apertures 325 and the second hub 333 diameter are selected so that the disk 330 may rotate freely within the plate assembly 320 while minimizing a gap between the disk 330 and the plate assembly 320. The plate assembly 320 allows limited axial movement of each of the plurality of disks 330 and allows unlimited rotational movement of each of the plurality of disks 330 while holding the disks 330 between the first portion 322 and the second portion 324 of the plate assembly. Radial movement of the disks 330 may be limited by the diameter of the collar 336 and the diameter of the bore of the first portion 322. The disks 330 are described below in greater detail in connection with FIGS. 10, 12A, and 12B.

FIG. 9D illustrates an end view of the drape plate assembly 300 including the frame assembly 310 with the first portion 312, the first channel 315, the second channel 315′, the bosses 326 of plate assembly 320, the disks 330, and the electrical connector 350. The first channel 315 and the second channel 315′ are configured to receive the cassette 240.

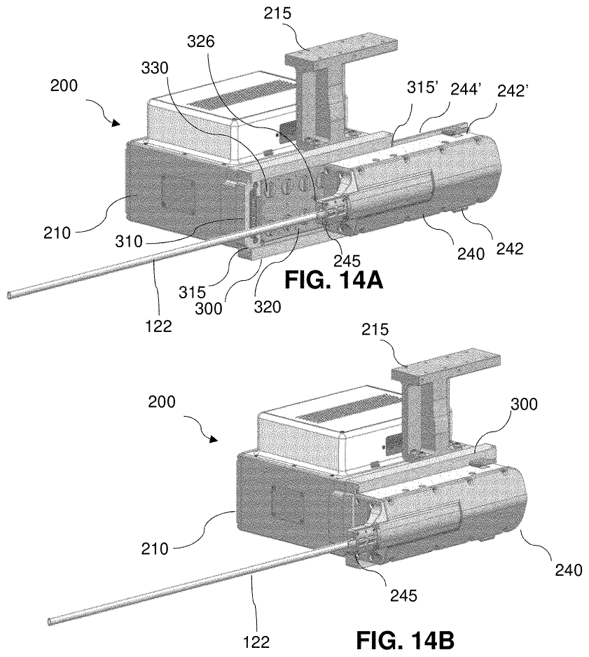

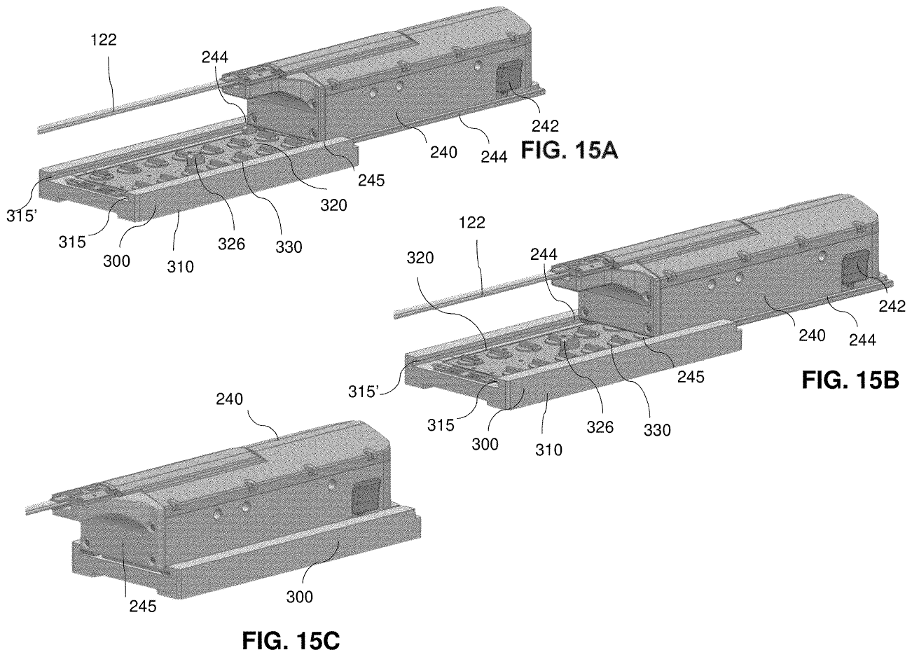

Specifically, a first rail 244 of the cassette 240 is fitted to the first channel 315 of the drape plate assembly 300 and the second rail 244′ of the cassette 240 is fitted to the second channel 315′ of the drape plate assembly, as described in detail below with respect to FIGS. 14A-14B and 15A-15C. The plurality of bosses 326 of the plate assembly 320 are exposed to receive the cassette 240. Specifically, a leading edge 245 of the cassette 240 impinges one or more of the plurality of bosses 326 of the plate assembly 320, causing the plate assembly 320 to move inwardly toward the motor couplings 222 via compression of the springs 327 and causing the plurality of coupling crowns 224 to retract so that the drape plate assembly 300 can receive the cassette 240. When the cassette 240 is in position, each of the plurality of coupling crowns 224 extend by the action of the corresponding spring 225 to mate each of the coupling crowns 224 with a respective one of the spooleys of the cassette 240.

FIG. 10A illustrates a detailed cutaway of a portion of the drape plate assembly 300 showing the first portion 322 and the second portion 324 of the plate assembly 320, the second portion 314 of the frame assembly 310, the disks 330, the spring 327, and fasteners 318. The plate assembly 320 is supported by the spring 327 which rests on the second portion 314 of the frame assembly 310. The spring 327 is held between the first portion 322 of the plate assembly 320 and the second portion 314 of the frame assembly 310. This configuration allows the plate assembly 320 to be displaced toward the second portion 314, as the spring 327 is compressed, when force is applied to the place assembly 320 and, in particular, to the bosses 326 of the plate assembly 320. The springs 327 bias the plate assembly 320 and, in turn, disks 330, toward engagement with the cassette 240 when the cassette 240 is attached to the drape plate assembly 300 to ensure the bosses 326 remain in a pocket of the cassette 240.

In some embodiments, the springs 327 may assist in biasing the disks 330 toward engagement with the cassette 240. In such embodiments, the springs 225 of the motor couplings 222 may also engage and bias the disks 330 toward engagement with the cassette 240.

FIG. 10B illustrates an exploded view of the drape plate assembly 300. The drape plate assembly 300 includes the electrical connector 350. The drape plate assembly 300 further includes the first portion 312 of the frame assembly 310 with the first channel 315 and the second channel 315′. The drape plate assembly 300 further includes the first portion 322 of the plate assembly 320 with bosses 326 and a plurality of apertures 323. The drape plate assembly 300 further includes the disks 300, each with the first side 331, the second side 331′ opposite to the first side 331, and the collar 336 between the first side 331 and the second side 331′. The first side 331 includes the first hub 332 and the first mating feature 334 and the second side 331′ includes the second hub 333 and the second mating features 335. The drape plate assembly 300 further includes the first second portion 324 of the plate assembly 320 with the plurality of apertures 325. The drape plate assembly 300 further includes the second portion 314 of the frame assembly 310. The drape plate assembly 300 further includes a plurality of springs 327. When assembled, the frame assembly 310 holds the plate assembly 320 with the plurality of disks 330 held within the plurality of apertures 323, 325 of the plate assembly 320. Specifically, the collars 336 of the disks 330 are held between the first portion 322 and the second portion 324 of the plate assembly 320 while the first hubs 332 and second hubs 333 are fitted with the apertures 323, 325. This configuration allows unlimited rotation of the disks 330 while the disks 330 are held in position relative to the plate assembly 320 and allows each of the disks 330 limited axial movement or “float” for individual engagement with a respective one of the coupling crowns 224. Further, the springs 327 permit the plate assembly 320 to float within the frame assembly 310 to allow the drape plate assembly to be connected to the drive unit 200 and to allow the cassette 240 to be fitted to the drape plate assembly 300.

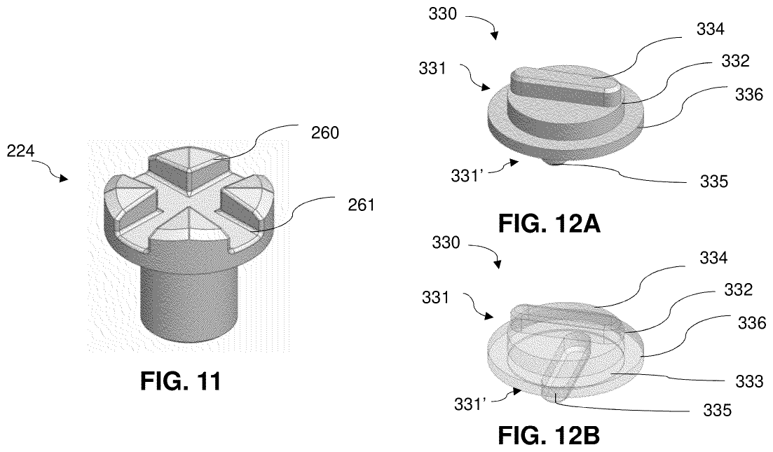

The disk 330, its placement within the plate assembly 320, and its interaction with coupling crown 224 is further described with reference to FIG. 11, FIG. 12A, and FIG. 12B.

FIG. 11 illustrates the coupling crown 224 of the drive unit 200 with the coupling crown mating feature 260.

FIG. 12A illustrates the disk 330 and FIG. 12B illustrates the disk 330 that is partially transparent to provide a view of additional features of the disk 330. In some embodiments, the disk 330 includes a first hub 332 on a first side 331 of the disk 330, a second hub 333 on a second side 331′ of the disk 330 opposite the first side 331 and a collar 336 extending radially outward between the first side 331 and the second side 331′ of the disk 330. The first hub 332 includes a first mating feature 334. The second hub 333 includes a second mating feature 335.

Each of the disks 330 is configured to engage with the coupling crown 224 of the motor coupling 222 of the drive unit 200 of the surgical robotic device 10. Each of the motor couplings 222 includes the coupling crown 224 with the coupling crown mating feature 260 that couples with one of the first or the second mating feature 334, 335 of the disk 330. The spooleys of the cassette 240 may include a similar mating feature that couples with one of the first or the second mating feature 334, 335 of the disk 330. By way of example, the first mating feature 334 may be coupled to the spooley and the second mating feature 335 may be coupled to the coupling crown mating feature 260. Those of skill in art will appreciate that the mating features may be swapped such that the first mating feature 334 may be coupled to the coupling crown mating feature 260 and the second mating feature 335 may be coupled to the spooley.

In some embodiments, the first mating feature 334 includes a first rectangularly shaped bar extending along a surface of the first hub 332 through a central portion of the surface of the first hub 332, and the second mating feature 335 includes a second rectangularly shaped bar extending along a surface of the second hub 333 through a central portion of the surface of the second hub 333. As shown in FIG. 12B, the first mating feature 334 and the second mating feature 335 are offset from each other at approximately ninety degrees. Each of the first mating feature 334 and the second mating feature 335 include chamfered edges on the rectangularly shaped bars. Notably, the present disclosure is not limited to this design and can be varied to mate with the coupling crown 224.

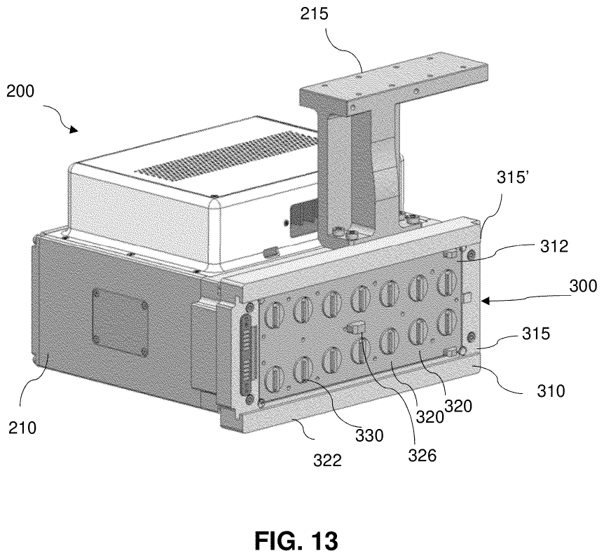

During an engagement process, after the drape plate assembly 300 has been fitted to the drive unit 200 (described below in connection with FIG. 13), the motor couplings 222 may be driven to rotate the crown mating feature 260 until the first or the second mating feature 334, 335 engages the coupling crown mating feature 260 (i.e., one of the mating features 334, 335 seats in one of the channels 261). In some embodiments, the first side 331 and the second side 331′ may be interchangeable such the disk 330 may be inserted into the plate assembly 320 in either orientation. The angled or chamfered surfaces of the second mating feature 335 and the coupling crown mating feature 260 facilitate mating of the second mating feature 335 with the coupling crown mating feature 260 during rotation of the coupling crown mating feature 260. When the first or second mating feature 334, 335 is mated to the coupling crown mating feature 260, the first or second mating feature 334, 335 rests within one of the channels 261 of the coupling crown mating feature 260. The configuration of the channels 261 provides the advantage that the first or second mating feature 334, 335 may mate with either of the two channels 261 and may accommodate some misalignment and/or function even if mated imperfectly. For example, the mating of the first or second mating feature 334, 335 and the coupling crown mating feature 260 may accommodate angular and/or axial misalignment or parallel misalignment. For example, the first or second mating feature 334, 335 may extend partially beyond the channels 261, while still providing sufficient engagement for cooperation of the first or second mating feature 334, 335 and the coupling crown mating feature 260. In some embodiments, the first or second mating feature 334, 335 and the coupling crown mating feature 260 constitute a portion of an Oldham-style coupling. Additionally, the chamfered or angled surfaces of the first or second mating feature 334, 335 and the coupling crown mating feature 260 facilitate mating of the first or second mating feature 334, 335 and the coupling crown mating feature 260.

The first or second mating feature 334, 335 and the coupling crown mating feature 260 may be mated during an initialization procedure as the coupling crown mating feature 260 is rotated. In particular, during the initialization procedure, the coupling crown mating feature 260 may be spun by the drive unit 200 until the first or second mating feature 334, 335 mates with the coupling crown mating feature 260. In some embodiments, the drive motor 220 may spin the coupling crown 224 until the first or second mating feature 334, 335 and the coupling crown mating feature 260 are aligned.

In order to place the drape plate assembly 300 onto the drive unit 200, the drape plate assembly 300 is coupled to the motor mount plate 230. In some embodiments, the drape plate assembly 300 may be slidably engaged with the motor mount plate 230. In some embodiments, the drape plate assembly 300 may be coupled to the motor mount plate 230 with features such as a fasteners (e.g., machine screw or bolt), a snap-fit assembly, or other mechanical joint.

During the coupling process, the coupling crowns 224 retract via forces placed thereon by the coupling action. The forces applied to the coupling crowns 224 compress the springs 225 of the motor couplings 222 to permit coupling of the drape plate assembly 300 and the motor mount plate 230. As discussed above in connection with the disk 330, the plurality of drive motors 220 may then turn each of the plurality of motor couplings 222 to rotate the corresponding coupling crown 224 and engage the coupling crown mating feature 260 with the first or second mating feature 334, 335 of a corresponding one of the plurality of disks 330.

FIG. 13 illustrates the drape plate assembly 300 coupled to the motor mount plate 230 of the drive unit 200. The drive unit 200 includes the drive unit housing 210 and the carriage attachment mount 215. With respect to the drape plate assembly 300, the first portion 322 of the plate assembly 320 and the first portion 312 of the frame assembly 310 are exposed or positioned outwardly with respect to the drive unit 200. The first channel 315 and the second channel 315′ of the frame assembly 310 are exposed to receive the cassette 240.

Additionally, the plurality of bosses 326 of the plate assembly 320 are exposed to receive the cassette 240. In some embodiments, the surgical robotic system 10 includes three drape plates 300, one for each of the robotic arms 42 and one for the camera assembly 44. Nonetheless, those skilled in the art will appreciate that some surgical robots may have more than three drape plates 300 or fewer drape plates 300. Likewise, those skilled in the art will appreciate that the number of apertures and corresponding disks illustrated in the drape plate assembly 300 can change depending on the number of spooleys or other elements that need motorized actuation.

Installation of the cassette 240 onto the drape plate assembly 300 may be better understood by reference to FIG. 14A-14B and 15A-15C. FIGS. 14A and 14B illustrate the attachment of the cassette 240 to the drape plate assembly 300 when the drape plate assembly 300 is mounted on the drive unit 200. FIG. 15A-15C illustrate the attachment of the cassette 240 to the drape plate assembly 300 when the drape plate assembly 300 is independent of the drive unit 200.

The cassette 240 includes a first rail 244 and a second rail 244′ which extend from opposing lower sides of the cassette 240 and extend substantially along the length of the cassette 240. The cassette 240 includes a first side button 242 and a second side button 242′. In some embodiments, the first side button 242 and the second side button 242′ are used to lock the cassette 240 into position on the drape plate assembly 300 and to unlock the cassette 240 from the drape plate assembly 300 so that it may be removed from the drape plate assembly 300. In some embodiments, the cassette 240 may lock into engagement with the drape plate assembly 300 without operation of the first side button 242 and the second side button 242′, but the first side button 242 and the second side button 242′ may be used to release the cassette 240 from the drape plate assembly 300 in order to remove the cassette 240. A shaft 122 extends from the cassette 240. A plurality of spooleys (not shown) are positioned within cassette 240. The spooleys are driven by the plurality of drive motors 220 via the disks 330 when to the drape plate assembly 300 is mounted to the drive unit 200 and the cassette 240 is mounted to the drape plate assembly 300. The spooleys may, in turn, control one or more instruments or surgical tools positioned on the shaft 122 for use in a surgical procedure.

The cassette 240 is attached to the drape plate assembly 300 by fitting the first rail 244 of the cassette 240 into the first channel 315 of the drape plate assembly 300 and fitting the second rail 244′ of the cassette 240 into the second channel 315′ of the drape plate assembly. Then, cassettes 240 may be slid into position onto the drape plate assembly 300. As the cassette 240 slides into position on the drape plate assembly 300, a leading edge 245 of the cassette 240 impinges one or more of the plurality of bosses 326 of the plate assembly 320, causing the plate assembly 320 to move inwardly toward the motor couplings 222 via compression of the springs 327 and causing the plurality of coupling crowns 224 to retract.

The movement of the drape plate assembly 300 causes sufficient compliance to permit the cassette 240 to slide into position, while the outwardly bias of the plurality of coupling crowns 224 by the springs 225 causes the plate assembly 320 and the plurality of disks 330 to shift back to the extended position so that the first or the second mating feature 334, 335 of the disks 330 is in position to engage with a complementary mating surface of the cassette 240 to drive a spooley (not shown).

With the cassette 240 attached to the drape plate assembly 300 and the drape plate assembly 300 attached to the drive unit 200, the drive motors 220 may drive the spooleys of the cassette 240 via the disks 330 which engage both the spooleys and the coupling crown 224 of the motor coupling 222 with the disks 330. In some embodiments, engaging the first or second mating feature 334, 335 with the spooley may be substantially similar to the process for engaging the first or second mating feature 334, 335 and the coupling crown mating feature 260. Accordingly, the drive unit 200 delivers torque to the cassette 240 while the drape plate assembly 300 provides a sterile barrier between the cassette 240 and the drive unit 200.

1. A drape plate assembly for a mechanical drive of a surgical robotic device, the drape plate assembly comprising:
a frame assembly having a first portion mateable to a second portion;
a plate assembly having a first portion mateable to a second portion, each of the first portion and the second portion of the plate assembly having a plurality of apertures, the plate assembly disposable between the first portion and the second portion of the frame assembly; and
a plurality of disks disposable between the first portion and the second portion of the plate assembly, each of the plurality of disks disposable in a respective one of the plurality of apertures in the first portion of the plate assembly and in a respective one of the plurality of apertures in the second portion of the plate assembly.

2. The drape plate assembly of claim 1, wherein the first portion of the plate assembly includes one or more bosses on a surface of the first portion of the plate assembly facing the first portion of the frame assembly.

3. The drape plate assembly of claim 1, wherein the first portion of the frame assembly includes a first channel on a first longitudinal side of the first portion of the frame assembly and a second channel on a second longitudinal side of the first portion of the frame assembly that is opposite the first longitudinal side, and wherein the first channel and the second channel allow slidable mating with a cassette.

4. The drape plate assembly of claim 1, further comprising an interface circuit board mounted to the frame assembly and configured to couple with a second interface circuit board of the surgical robotic device.

5. The drape plate assembly of claim 1, further comprising a plurality of springs disposed between the first portion of the plate assembly and the second portion of the frame assembly permitting compression of the plate assembly within the frame assembly.

6. The drape plate assembly of claim 1, wherein each of the plurality of disks comprises:
a first hub on a first side of the disk, the first hub including a first mating feature;
a second hub on a second side of the disk opposite the first side, the second hub including a second mating feature; and
a collar extending radially outward between the first side of the disk and the second side of the disk.

7. The drape plate assembly of claim 6, wherein each of the plurality of disks is configured to engage with a motor coupling of an drive unit of the surgical robotic device.

8. The drape plate assembly of claim 7, wherein each of the motor couplings comprises a coupling crown with a coupling crown mating feature that couples with the first mating feature of the first hub.

9. The drape plate assembly of claim 6, wherein the collar has a diameter greater than either of a first hub diameter of the first hub and a second hub diameter of the second hub.

10. The drape plate assembly of claim 6, wherein each of the apertures in the first portion of the plate assembly has a first diameter that is smaller than the diameter of the collar and larger than the first hub diameter and wherein each of the plurality of apertures in the second portion of the plate assembly has a second diameter that is smaller than the diameter of the collar and larger than the second hub diameter.

11. The drape plate assembly of claim 10, wherein the first diameter and the first hub diameter are selected so that the disk may rotate freely within the plate assembly while minimizing a gap between the disk and the plate assembly.

12. The drape plate assembly of claim 1, wherein the plate assembly allows limited axial movement of each of the plurality of disks and allows unlimited rotational movement of each of the plurality of disks while holding the disks between the first portion and the second portion of the plate assembly.

13. The drape plate assembly of claim 10, wherein the second diameter and the second hub diameter are selected so that the disk may rotate freely within the plate assembly while minimizing a gap between the disk and the plate assembly.

14. The drape plate assembly of claim 6,
wherein the first mating feature includes a first rectangularly shaped bar extending along a surface of the first hub through a central portion thereof, and
wherein the second mating feature includes a second rectangularly shaped bar extending along a surface of the second hub through a central portion thereof.

15. The drape plate assembly of claim 14, wherein the first rectangularly shaped bar and the second rectangularly shaped bar are offset from each other at approximately ninety degrees.

16. The drape plate assembly of claim 14, wherein the first rectangularly shaped bar includes one or more chamfered edges.

17. The drape plate assembly of claim 1, further comprising a connector mounted to the frame assembly to electrically connect a first portion of the surgical robotic device to a second portion.

18. A motor drive system for a surgical robotic system comprising:
a drive unit comprising:
a drive unit housing;
a plurality of drive motors within the drive unit housing;
a motor mount plate;
a plurality of motor couplings; and
a drape plate assembly according to any of claims 1-17.

19. A method of draping a surgical robotic device, the method comprising:
attaching a drape plate assembly according to any of claims 1-17 to a drive unit of the surgical robotic device;
providing drape film over one or more components of the surgical robotic device; and
heat sealing one or more edges of the drape film to the frame assembly of the drape plate assembly.
